(19) United States
(12) Patent Application Publication
(10) Pub. No.: US 2019/0134425 A1
(43) Pub. Date: May 9, 2019

(54) Methods and Systems for Tumor Tracking in the Presence of Breathing Motion

(71) Applicant: Mitsubishi Electric Research Laboratories, Inc., Cambridge, MA (US)

(72) Inventor: Jeroen van Baar, Arlington, MA (US)

(21) Appl. No.: 15/806,611

(22) Filed: Nov. 8, 2017

(51) Int. Cl. A61N 5/10

(52) U.S. Cl. CPC A61N 5/1049; A61N 2005/1061; A61N 5/1077; A61N 5/1067

(57) ABSTRACT

A real-time treatment system for tracking of an abnormal growth of tissue in a body of a patient. An offline stage includes storing historical image data of internal structures of bodies of different patients caused by a respiration of patients. A processor obtains for each patient image data, a sequence of dense motion vector fields. An online stage includes accepting an initial pair of temporally separated images of internal anatomical structures of the body of the patient during respiration in real-time. An imaging processor tracks changes of positions of landmark points over the initial pair of temporally separated images, to produce a first local motion representation of the body. An interpolation processor interpolates the first local motion representation to produce a first dense motion vector field for a controller to track a first location of the abnormal growth of tissue of the patient, to produce a first location.

### FIELD

The present disclosure relates generally to radiotherapy, and more particularly to tracking a motion of a pathological anatomy during respiration of a patient while delivering particle beam radiotherapy.

### BACKGROUND

Conventional radiation therapies of tumors are structured to accomplish two objectives, first removing of the tumor, and second preventing of damage to healthy tissue and organs-at-risk near the tumor. Most tumors can be removed completely if an appropriate radiation dose is delivered to the tumor. Unfortunately, delivering certain amounts of doses of radiation to eliminate a tumor can likely result in complications, due to damaging healthy tissue and organs-at-risk surrounding the tumor. One conventional technique to address this problem is a three-dimensional (3D) conformal radiation therapy that uses beams of radiation in treatment shaped to match the tumor to confine the delivered radiation dose to only the tumor volume defined by the outer surfaces of the tumor, while minimizing the dose of radiation to surrounding healthy tissue or adjacent healthy organs.

Typically, to perform these radiation treatment therapy plans, involves defining an exact type, locations, distribution and intensity of radiation sources so as to deliver a desired spatial radiation distribution. Radiation treatment therapy planning begins with a set of Computed Tomography (CT) images of a patient's body in the region of the tumor, i.e. CT images. Those methods assume that the patient is stationary. However, even if the patient is stationary, radiotherapy requires additional methods to account for the motion of the tumor due to respiration, in particular for treating a tumor located in or near the lungs of the patient, e.g., posterior to the sternum, or a tumor in the liver. Breath-holding and respiratory gating are two primary methods used to compensate for the motion of the tumor during the respiration, while the patient receives the radiotherapy.

A breath-hold method requires that the patient holds the breath at the same time and duration during the breathing cycle, e.g., a hold of 20 seconds after completion of an inhalation, thus treating the tumor as stationary. A respirometer is often used to measure the rate of respiration, and to ensure the breath is being held at the same time in the breathing cycle. Such a method can require training the patient to hold the breath in a predictable manner, which is often difficult given the health condition of the patient.

Respiratory gating is the process of turning the beam on and off as a function of the breathing cycle. The radiotherapy is synchronized to the breathing pattern, limiting the radiation delivery to a specific time-span of the breathing cycle and targeting the tumor only when the location of the tumor in a predetermined range. The respiratory gating method is usually quicker than the breath-hold method, but requires the patient to have many sessions of training to breathe in the same manner for long periods of time. Such training can require days of practice before treatment can begin. Also, with the respiratory gating some healthy tissue around the tumor can be irradiated to ensure complete treatment of the tumor.

Attempts have been made to avoid the burden placed on a patient treated by the breath-hold and respiratory gating methods. For example, one method tracks the motion of the tumor during the respiration, see, e.g., US Published Application 2012/0226152 A1 filed Mar. 3, 2011. However, tracking the motion of the tumor without placing internal imaging markers near the tumor and/or organs of the patient is difficult.

Other problems with these methods are challenged due the characteristics of the imaging modalities which can acquire internal anatomical images, locating the tumor from said images is a difficult task. A major problem is the fact the tumor is poorly visible (where poorly includes the case where the tumor is entirely invisible), due to the tissue properties of the tumor, or due to masking by bone structures such as ribs and spinal cord.

One challenge during the delivery of radiation to a patient for treating a pathological anatomy, such as a tumor or a lesion, is identifying a location of the tumor. The most common localization methods use an x-ray to image the body of the patient to detect the location of the tumor.

### SUMMARY

The present disclosure relates to tracking a motion of a pathological anatomy during respiration of a patient while delivering particle beam radiotherapy. In particular, the present disclosure tracks a location of a physical anomaly in a body of a patient, such as a tumor, in the anatomical areas affected by motion caused by respiration of the patient.

Some embodiments of present disclosure are based on a realization that if a dense motion vector field between the two consecutive medical images of the internal anatomical structure of the body of the patient is determined, the motion vectors in the dense motion vector field describe the motion for every point of the internal anatomical structure. To better understand what a dense motion vector field is, it is first important to know what is a motion vector field. In computer vision, the motion vector field is referred to an ideal representation of 3D motion as it is projected onto a camera image. Given a simplified camera model for example, each point in the image is the projection of some point in the 3D scene but the position of the projection of a fixed point in space can vary with time. The motion vector field can formally be defined as the time derivative of the image position of all image points given that they correspond to fixed 3D points. This means that the motion vector field can be represented as a function which maps image coordinates to a 2-dimensional vector. To that end, the motion vector field capturing the motion of all points in the image is referred herein as a dense motion vector field.

Regarding our realization that if a dense motion vector field between the two consecutive medical images of the internal anatomical structure of the body of the patient is determined, the motion vectors in the dense field describe the motion for every point of the internal anatomical structure. It is further important to know when the internal anatomical structure includes a tumor, the dense motion vector field describes the motion of the tumor even if the tumor is poorly visible in those medical images. Thus, our realization can overcome the conventional challenges of solving the problem from tracking poorly or invisible tumors, which undergo breathing motion. However, determining the dense motion vector field is a computationally intensive task making such a determination ill-suited for real-time processing required for irradiation of the tumor during respiration of the patient, which is another challenged that needed to be addressed.

Some embodiments are based on understanding medical images do include points forming traceable landmarks. For example, corners of bones, or internal organs of the body, or specific tissue patterns can be detected as landmarks in consecutive frames of medical imaging and the changes in the position of those landmarks provide local information on the 2-D motion of the internal anatomical structure of the patient. Specifically, the landmarks tend to be sparse and distributed over the internal anatomical structure of the patient. Local motion information of the internal anatomical structure of the patient is directly derived from position changes of landmarks in consecutive images. The motion of internal anatomical structures of the patient is not uniform, but rather depends on the specific location in the patient's body. Provided that one can obtain a reasonable number of landmarks and associated local motion information, the local motion information can be interpolated to produce dense motion information, i.e., a dense motion vector field. For example, given a number of landmarks that cover the area of the patients' lungs, the local motion can be interpolated to provide dense motion vector field for the entire lung. However, naively selected interpolation techniques can produce inaccurate results.

Some embodiments are based on a realization that such an interpolation can be performed online by using past data or historical data of motion of internal anatomical structures of different patients caused by respiration of the patients. The historical data can be used to obtain a compact representation of the motion of internal anatomical structures of the different patients caused by respiration of the patients. This obtained representation is learned offline based on measurements of the breathing of many patients, and then used online, or in real-time, to interpolate local motions of a specific patient undergoing irradiation.

For example, in one embodiment, the representation can be defined by motion vector bases captured by bases vectors. Each bases vector represents a component in the space of possible breathing motions, and each breathing motion can be expressed as a combination of bases vectors. Wherein in one embodiment, the set of bases vectors are determined from a principal component analysis (PCA) on dense motion vector fields. In one embodiment, the dense motion vector fields are determined using an Anisotropic Huber-L1 Optical Flow method. Note, it is contemplated other methods can be used, such as edge-preserving patch-based motion (EPPM) to determine the dense motion vector fields. This embodiment empirically adjusts the parameters to obtain dense motion vector fields, which can both reduce an error between a warped source and target images, but still provide reasonably smooth motion vector fields.

In one embodiment, the interpolation of local motion information using such a representation of motion bases vectors, produces a weighted combination of the bases vectors forming a dense motion vector field with optimum approximation of the local motion vectors. In other words, the interpolation determines such a dense motion vector field which fits the local motion information determined by tracking the landmarks. Notably, the selection of landmarks is of lesser importance, and landmarks for different pairs of consecutive medical images can vary.

In such a manner, the tumor tracking problem is divided into an offline learning stage, as noted above, and an online sparse reconstruction stage. During the offline stage, some embodiments apply dimensionality reduction to the motion vectors of respiration in fluoroscopic (X-ray) video to enable reconstruction of the tumor motion from a set of matched landmarks in the online stage. To handle the motion of different anatomical features in fluoroscopic imagery, some embodiments can apply a subspace decomposition to the imagery prior to determining landmarks.

As a result, the embodiments provide for tumor tracking methods which handle poor tumor visibility (including invisibility of tumors), handle noisy imagery and incorporate biomarkers without any necessary modifications. Since the dense motion is interpolated from local motion, motion information is computed even for poorly visible tumors. Interpolation from local motion provides a certain amount of robustness to noisy imagery. Finally, since biomarkers provide landmarks, the biomarkers landmarks provide additional local motion information. In addition, the embodiments provide a practical approach to tumor tracking, which can be implemented on modern Graphics Processing Units (GPUs) hardware for real-time performance.

Methods and systems of the present disclosure can facilitate radiotherapy during any phase of the respiration of the patient, along with minimizing tumor position uncertainty during the treatment. At least one aspect of the present disclosure includes determining the location of the tumor without the need to use invasive imaging markers inside the body of the patient.

Due to the characteristics of the imaging modalities of medical images that can acquire internal anatomical structure including the tumor, locating the tumor from those medical images is a difficult task. One problem for such a difficulty lies in the fact that the tumor is poorly visible on those images, due to the tissue properties of the tumor, or due to masking by bone structures such as ribs and spinal cord. In other words, pixels corresponding to the tumor in the medical images do not possess characteristics making them traceable landmarks that can be relatively easily detected in the sequence of medical images.

According to an embodiment of the present disclosure, a real-time treatment system for tracking of an abnormal growth of tissue in a body of a patient over a treatment period. The real-time treatment system including a memory to store in an offline stage, historical image data of internal anatomical structures of bodies of different patients caused by a respiration of the patients. A processor in communication with the memory during the offline state, is configured, to obtain for each patient image data, a sequence of dense motion vector fields. Wherein the sequence of dense motion vector fields of all the patients is transformed into a set of learned bases vectors of breathing motion and stored in the memory. An imaging interface of an online stage accepts an initial pair of temporally separated images of internal anatomical structures of the body of the patient during respiration in real-time. An imaging processor in communication with the imaging interface, to track changes of positions of landmark points over the initial pair of temporally separated images, to produce a first local motion representation of the body of the patient, caused by the respiration of the patient. An interpolation processor in communication with the memory and the imaging processor, interpolates the first local motion representation by accessing the memory, and using the stored set of learned bases vectors of breathing motion, to produce a first dense motion vector field of the body of the patient. A controller in communication with interpolation processor, to track a first location of the abnormal growth of tissue of the patient, according to the produced first dense motion vector field, to produce a first location of the abnormal growth of tissue of the patient. Iteratively, accepting in real-time pairs of temporally separated images of internal anatomical structures of the body of the patient during respiration for each iteration, to produce a dense motion vector field of the body of the patient, to track a location of the abnormal growth of tissue of the patient from the first location, or a previously determined location, according to each produced dense motion vector field, and ending the iteration, at an end of the treatment period.

According to another embodiment of the present disclosure, a real-time treatment method including the steps of storing in a memory in an offline stage, historical image data of internal anatomical structures of bodies of different patients caused by a respiration of the patients. Obtaining for each patient image data via a processor in communication with the memory during the offline stage, a sequence of dense motion vector fields. Wherein the sequence of dense motion vector fields of all the patients is transformed into a set of learned bases vectors of breathing motion and stored in the memory. Wherein the set of bases vectors is an output of a principal components analysis (PCA) on the dense motion vector fields. Accepting an initial pair of temporally separated images of internal anatomical structures of the body of the patient during respiration in real-time via an imaging interface of an online stage. Tracking changes of positions of landmark points over the initial pair of temporally separated images, to produce a first local motion representation of a body of a patient, caused by the respiration of the patient, via an imaging processor in communication with the imaging interface. Interpolating the first local motion representation by accessing the memory, and using the stored set of learned bases vectors of breathing motion, to produce a first dense motion vector field of the body of the patient over a treatment period, via an interpolation processor in communication with the memory and the imaging processor. Tracking a first location of an abnormal growth of tissue of the patient, according to the produced first dense motion vector field, to produce a first location of the abnormal growth of tissue of the patient, via a controller in communication with interpolation processor. Iteratively, accepting in real-time pairs of temporally separated images of internal anatomical structures of the body of the patient during respiration for each iteration, to produce a dense motion vector field of the body of the patient, to track a location of the abnormal growth of tissue of the patient from the first location, or a previously determined location, according to each produced dense motion vector field, and ending the iteration, at an end of the treatment period.

According to another embodiment of the present disclosure, a real-time treatment delivery system for tracking and irradiation of a tumor in a body of a patient over a treatment period. The real-time treatment delivery system including a memory to store in an offline stage, historical patient image data of a breathing motion of bodies of different patients, caused by a respiration of the patients. A processor in communication with the memory during the offline state, is configured, to obtain for each stored historical patient image data, a sequence of dense motion vector fields. Wherein the sequence of dense motion vector fields of all the patients is transformed into a set of learned bases vectors of breathing motion, and stored in the memory. An imaging interface of an online stage accepts an initial pair of temporally separated images of internal anatomical structures of the body of the patient positioned for the irradiation, during respiration in real-time. An imaging processor in communication with the imaging interface, to track changes of positions of landmark points over the initial pair of temporally separated images, to produce a first local motion information of the body of the patient, caused by the respiration of the patient. An interpolation processor in communication with the memory and the imaging processor, interpolates the first local motion information by accessing the memory, and using the stored set of learned bases vectors of breathing motion, to produce a first dense motion vector field of the body of the patient. An accelerator to produce a particle beam suitable for radiotherapy of the patient. A controller in communication with interpolation processor, to track a first location of the tumor of the patient, according to the produced first dense motion vector field, to produce a first location of the tumor of the patient. Iteratively, accepting in real-time pairs of temporally separated images of internal anatomical structures of the body of the patient during respiration for each iteration, to produce a dense motion vector field of the body of the patient, to track a location of the tumor of the patient from the first location, or a previously determined location, according to each produced dense motion vector field, and ending the iteration, at an end of the treatment period.

### BRIEF DESCRIPTION OF THE DRAWINGS

The presently disclosed embodiments will be further explained with reference to the attached drawings. The drawings shown are not necessarily to scale, with emphasis instead generally being placed upon illustrating the principles of the presently disclosed embodiments.

FIG. 1A is a flow diagram illustrating a real-time treatment system for tracking of an abnormal growth of tissue in a body of a patient over a treatment period, according to embodiments of the present disclosure;

FIG. 1B is a schematic illustrating the system of FIG. 1A, implemented using some components of the system that can implement the system to a patient's body, according to embodiments of the present disclosure;

FIG. 1C is a block diagram illustrating the method of FIG. 1A, that summarizes the two main offline and online steps according to embodiments of the present disclosure;

FIG. 1D is a block diagram illustrating the method of FIG. 1A, showing a functional diagram of the offline learning stage and online stage feature and reconstruction steps, according to embodiments of the present disclosure;

FIG. 2A is a block diagram illustrating some steps involved to learn the motion bases vectors, according to embodiments of the present disclosure;

FIG. 2B is a schematic illustrating the concept of capturing of historical data of internal anatomical structures of patients, according to embodiments of the present disclosure;

FIG. 2C is a schematic illustrating the sequence of images over time, according to embodiments of the present disclosure;

FIGS. 2D and 2E illustrate graphs of a pair of temporally separated images (t0, t1), such that FIG. 2D shows a graph of the frame at time t0 and FIG. 2E shows a graph of the frame at t1, in a sequence of time, according to embodiments of the present disclosure;

FIG. 2F illustrates a graph of the computed motion vectors for the pair of temporally separated images (t0—FIG. 2D and t1—FIG. 2E) that illustrates the motion vectors (or flow) from t0 of FIG. 2D to t1 of FIG. 2E, showing the direction and magnitude, according to embodiments of the present disclosure;

FIG. 3A is a flow diagram illustrating some steps involved in the motion field reconstruction system to determine motion vectors for updating the tumor location, according to embodiments of the present disclosure;

FIG. 3B is a schematic illustrating the concept of capturing real-time data of internal anatomical structures of the patient, according to embodiments of the present disclosure;

FIG. 3C is a schematic illustrating the sequence of images t0, t1, t2 over time, according to embodiments of the present disclosure;

FIGS. 3D and 3E illustrate graphs of an initial pair of temporally separated images (t0, t1), such that FIG. 3D shows a graph of the frame at time t0 and FIG. 3E shows a graph of the Frame at t1, in a sequence of time, to produce a local motion representation, according to embodiments of the present disclosure;

FIG. 3F illustrates a sketch for reconstruction of a dense motion field, according to embodiments of the present disclosure;

FIG. 4A is a graph illustrating, a tumor located within the reconstructed dense motion field according to embodiments of the present disclosure;

FIG. 4B is a graph illustrating, a region selection of dense motion vectors near a tumor location according to embodiments of the present disclosure;

FIG. 4C is a graph illustrating, the averaging of the dense motion vectors within a region selection near a tumor location according to embodiments of the present disclosure;

FIG. 4D is a graph illustrating, updating the tumor location in a next frame according to the averaged dense motion vector of FIG. 4C according to embodiments of the present disclosure; and

FIG. 5 is a block diagram illustrating the methods of FIG. 1A and FIG. 1B, that can be implemented using an alternate computer or processor, according to embodiments of the present disclosure.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The following description provides exemplary embodiments only, and is not intended to limit the scope, applicability, or configuration of the disclosure. Rather, the following description of the exemplary embodiments will provide those skilled in the art with an enabling description for implementing one or more exemplary embodiments. Contemplated are various changes that may be made in the function and arrangement of elements without departing from the spirit and scope of the subject matter disclosed as set forth in the appended claims.

Specific details are given in the following description to provide a thorough understanding of the embodiments. However, understood by one of ordinary skill in the art can be that the embodiments may be practiced without these specific details. For example, systems, processes, and other elements in the subject matter disclosed may be shown as components in block diagram form in order not to obscure the embodiments in unnecessary detail. In other instances, well-known processes, structures, and techniques may be shown without unnecessary detail in order to avoid obscuring the embodiments. Further, like reference numbers and designations in the various drawings indicated like elements.

Also, individual embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process may be terminated when its operations are completed, but may have additional steps not discussed or included in a figure. Furthermore, not all operations in any particularly described process may occur in all embodiments. A process may correspond to a method, a function, a procedure, a subroutine, a subprogram, etc. When a process corresponds to a function, the function's termination can correspond to a return of the function to the calling function or the main function.

Furthermore, embodiments of the subject matter disclosed may be implemented, at least in part, either manually or automatically. Manual or automatic implementations may be executed, or at least assisted, through the use of machines, hardware, software, firmware, middleware, microcode, hardware description languages, or any combination thereof. When implemented in software, firmware, middleware or microcode, the program code or code segments to perform the necessary tasks may be stored in a machine readable medium. A processor(s) may perform the necessary tasks.

### Definition of Terms

According to the definition of terms with regard to the present disclosure, the term “Radiation therapy” is considered the treatment of medical diseases by the application of ionizing radiation, for example ion beams, alpha emitters, x-rays or gamma rays. An example application of radiation therapy is the treatment of cancer using beams of ions, such as protons or carbon ions, such that the cancer cells are killed by the dose of radiation while adjacent healthy tissue is spared. The dense motion vector fields are also called labeled flows, flow fields, optical flow fields, or optical flow, and computing dense motion vector fields is referred to as computing optical flow or simply flow.

Overview

The present disclosure relates to tracking a motion of a pathological anatomy during respiration of a patient while delivering particle beam radiotherapy. In particular, the present disclosure tracks a location of a physical anomaly in a body of a patient, such as a tumor, in the anatomical areas affected by motion caused by respiration of the patient.

In particular, systems and methods of the present disclosure can be used for tracking tumors in lung fluoroscopic videos, where the tumors undergo motion due to respiration by the patient. Embodiments of the present disclosure employ an offline step for computing dense motion vector fields between consecutive frames of fluoroscopic X-ray video, and perform principal components analysis (PCA) over all dense motion vector fields to obtain a set of flow bases. In other words, the offline stage computes a set of basis vectors representing the breathing motion, such that these basis vectors are collected from various patients as historical data stored in a memory.

An online step includes determining feature matches between consecutive frames and solve an optimization problem to reconstruct a dense motion vector field between the frames. The dense motion vector field is used to determine the tumor location in a next frame of video. In other words, once the basis vectors are captured, the online stage reconstructs dense motion vector fields from feature correspondences using the basis vectors. Since there are only a small number of motion vector bases (compared to a large number of frames from which the flow bases are determined), reconstruction can be performed quickly.

The present disclosure is based on several realizations, that led to the development of the embodiments of the present disclosure. For example, some embodiments are based on a realization that if a dense motion vector field between the two consecutive medical images of the internal anatomical structure of the body of the patient is determined, the motion vectors in the dense field describe the motion for every point of the internal anatomical structure. To better understand what a dense motion vector field is, it is first important to know what is a motion field. In computer vision, the motion field is referred to an ideal representation of 3D motion as it is projected onto a camera image. Given a simplified camera model for example, each point in the image is the projection of some point in the 3D scene but the position of the projection of a fixed point in space can vary with time. The motion field can formally be defined as the time derivative of the image position of all image points given that they correspond to fixed 3D points. This means that the motion field can be represented as a function which maps image coordinates to a 2-dimensional vector. To that end, the motion field capturing the motion of all points in the image is referred herein as a dense motion vector field.

Regarding our realization if a dense motion vector field between the two consecutive medical images of the internal anatomical structure of the body of the patient is determined, the motion vectors in the dense field describe the motion for every point of the internal anatomical structure. It is further important to know when the internal anatomical structure includes a tumor, the dense motion vector field describes the motion of the tumor even if the tumor is poorly visible in those medical images. Thus, our realization overcomes the conventional challenges of solving the problem from tracking poorly or invisible tumors, which undergo breathing motion.

Some embodiments are based on understanding medical images do include points forming traceable landmarks. For example, corners of bones, or internal organs of the body, or specific tissue patterns can be detected as landmarks in consecutive frames of medical imaging and the changes in the position of those landmarks provide local information on the motion of the internal anatomical structure of the patient. Specifically, the landmarks tend to be sparse and distributed over the internal anatomical structure of the patient. Local motion of the internal anatomical structure of the patient is directly derived from position changes of landmarks in consecutive images. The motion of internal anatomical structures of the patient is not uniform, but rather depends on the specific location in the patient's body. Provided that one can obtain a reasonable number of landmarks and associated local motion information, the local motion information can be interpolated to produce dense motion information, i.e., a dense motion vector field. For example, given a number of landmarks that cover the area of the patients' lungs, the local motion can be interpolated to provide dense motion information for the entire lung.

Some embodiments are based on a realization that such an interpolation can be performed online by using past data or historical data of motion of internal anatomical structures of different patients caused by respiration of the patients. The historical data can be used to obtain a compact representation of the motion of internal anatomical structures of the different patients caused by respiration of the patients. This obtained representation is learned offline based on measurements of the breathing of many patients, and then used online, or in real-time, to interpolate local motions. For example, in one embodiment, the representation can be defined by motion vector bases captured by bases vectors. Each bases vector represents a component in the space of possible breathing motions, and each breathing motion can be expressed as a combination of bases vectors. Wherein in one embodiment, the set of bases vectors are determined from a principal component analysis (PCA) on dense motion vector fields. In one embodiment, the dense motion vector fields are determined using an Anisotropic Huber-L1 Optical Flow method. Note, it is contemplated other methods can be used, such as edge-preserving patch-based motion (EPPM) to determine the dense motion vector fields. This embodiment empirically adjusts the parameters to obtain dense motion vector fields, which can both reduce an error between a warped source and target images, but still provide reasonably smooth motion vector fields

In one embodiment, the interpolation of local motion information involves producing a weighted combination of the bases vectors to form a dense motion vector field with optimum approximation of the local motion vectors. In other words, the interpolation determines such a dense motion vector field which fits the local motion information determined by tracking the landmarks. Notably, the selection of landmarks is of lesser importance, and landmarks for different pairs of consecutive medical images can vary.

In such a manner, the tumor tracking problem is divided into an offline learning stage, as noted above, and an online sparse reconstruction stage. During the offline stage, some embodiments apply dimensionality reduction to the motion vectors of respiration in fluoroscopic video to enable reconstruction of the tumor motion from a set of matched features in the online stage. To handle the motion of different anatomical features in fluoroscopic imagery, some embodiments can apply a subspace decomposition to the imagery prior to determining features.

Some embodiments of the present disclosure can facilitate radiotherapy during any phase of the respiration of the patient, along with minimizing tumor position uncertainty during the treatment. At least one aspect of the present disclosure includes determining the location of the tumor without the need to use invasive imaging markers inside the body of the patient. However, it is possible for the systems and methods of the present disclosure to handle both markerless as well as marker-based tracking. If markers are present, the detected locations can simply be added to the sparse set of feature matches. By giving more weight to the marker locations in the optimization problem, we can ensure that solutions near those locations closely reflect the marker motion. The present disclosure overcomes the conventional challenges of poor tumor visibility and the practical requirement for fast computation.

In order to avoid the irradiation of healthy tissue, it is important to know where the tumor is located relative to the particle beam. Due to the characteristics of the imaging modalities of medical images that can acquire internal anatomical structure including the tumor, locating the tumor from those medical images is a difficult task. One problem for such a difficulty lies in the fact the tumor is poorly, if entirely not, visible on those images, due to the tissue properties of the tumor, or due to masking by bone structures such as ribs and spinal cord. In other words, pixels corresponding to the tumor in the medical images do not possess characteristics making them traceable landmarks that can be relatively easily detected in the sequence of medical images.

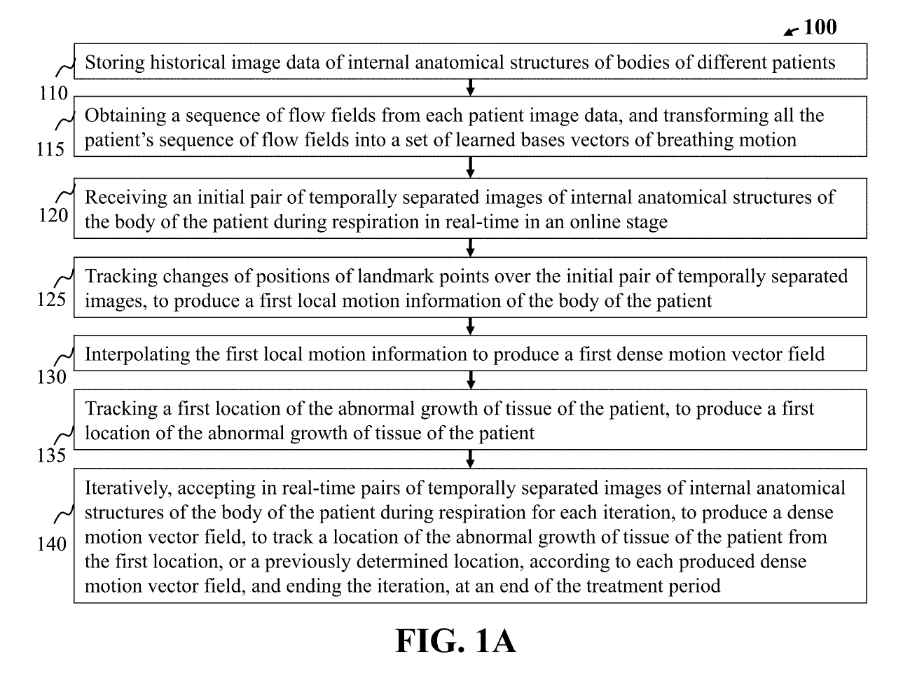

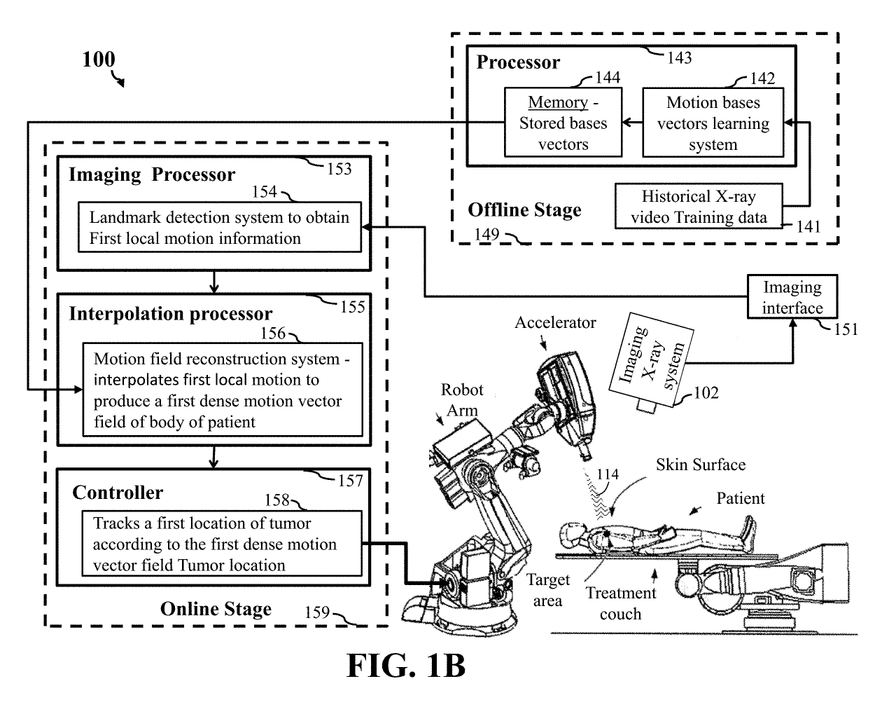

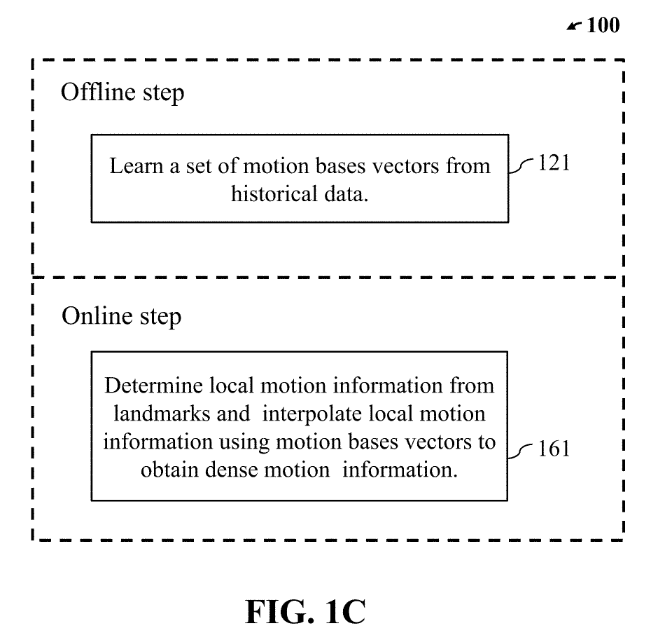

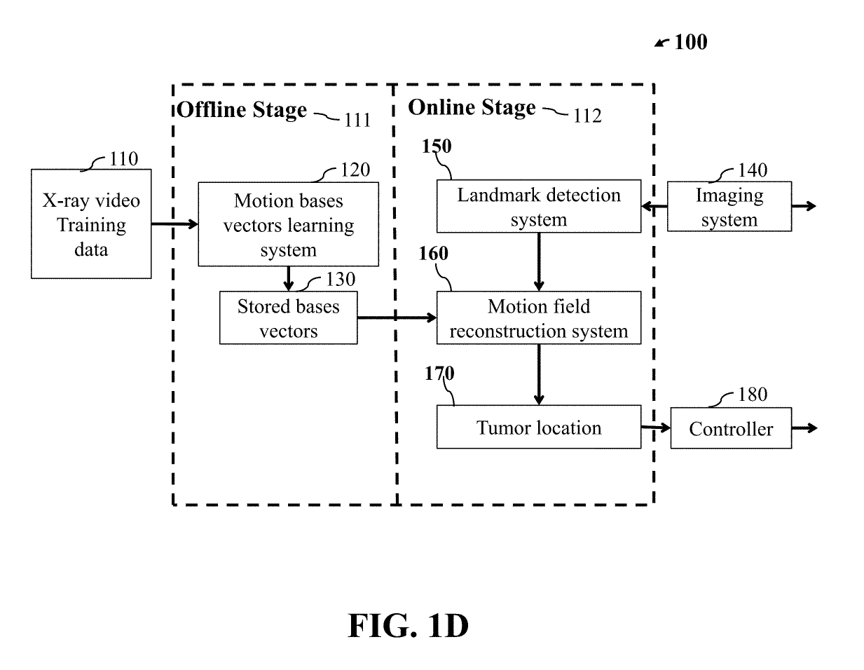

FIG. 1A is a block diagram illustrating a real-time treatment system for tracking of an abnormal growth of tissue in a body of a patient over a treatment period, according to embodiments of the present disclosure. Method 100 can track the location of a tumor over time in medical images during a treatment, according to embodiments of the present disclosure. In order to address the dimensionality reduction of motion vector fields for tumor tracking, the present disclosure uses sparse to dense motion vector fields. Computing dense motion vector fields can be computationally expensive. Rather than compute a motion vector for each pixel in an image (region), interpolating dense motion vector information from a sparse set of pixels instead can be computationally less intensive, and therefore faster, among other things. Simple linear interpolation would not be sufficient for achieving accurate results. The present disclosure shows that this interpolation can be performed according to a set of learned motion bases vectors. Which is referred to as the motion bases vectors.

Method 100 can consist of two steps: an offline step to learn the flow bases, and an online step to determine a sparse set of feature matches and reconstruct the dense flow field from those matches and flow bases. The dense flow field then provides the motion vectors necessary to determine the tumor motion between two consecutive frames of a fluoroscopic video. Initially, step 110 of method 100 is an offline step that includes storing historical image data of different patients.

Offline step 115 in FIG. 1A computes the dense motion vector fields (flow fields) for the historical data of patients and learn a bases vector representation of the breathing motion of all historical patients.

Online step 120 in FIG. 1A of receiving a pair of temporally separated images of a body of a patient, and determine landmarks, step 125, to produce a first local motion information of the internal anatomical structure of the patient; interpolating the first local motion information.

Online step 130 in FIG. 1A, to produce a first dense motion vector field; tracking a first location of abnormal growth by averaging the motion vectors in a neighborhood around the abnormal growth;

Online step 140 in FIG. 1A involving iterating steps 120, 125, 130 and 135 over the course of treatment.

FIG. 1B is a schematic illustrating the system of FIG. 1A, implemented using some components of the system that can implement the system to a patient's body, according to embodiments of the present disclosure.

FIG. 1B is a schematic illustrating the method of FIG. 1A, that can be implemented using a diagnostic system and radiation treatment system to a patient's body, according to embodiments of the present disclosure. The method 100 can be executed in an off-line stage 149 in at least one processor 143 that is in communication with historical data 141. The processor 143 learns motion bases vectors 142 from historical data 141. Furthermore, the processor 143 stores the learned motion bases vectors in memory 144. The method 100 can further be executed using at least one imaging interface 151, that is in communication with an imaging X-ray system 102, and generates medical image data of a patient, i.e., a body of a human or other living thing, positioned on a treatment couch. The method 100 can further be executed in an online stage 159 the imaging processor 153 determines landmarks and local motion information 154 from the images obtained from communication with the imaging interface 151. An interpolation processor 155 in communication with the imaging processor 153 reconstruct a dense motion vector field 156 from the local motion information 154 and stored motion bases vectors 144. The controller 157 in communication with the interpolation processor 155 determines 158 a location of the tumor from the dense motion vector field.

FIG. 1C is a block diagram illustrating the method of FIG. 1A, that illustrates the two main steps according to embodiments of the present disclosure. An offline step 121 which learns a set of motion bases vectors from historical data, and an online step 161 to determine a dense motion vector field from local motion information provided by a set of landmarks.

FIG. 1D shows a functional block diagram 100 illustrating the offline learning stage 111 and online stage 112 according to some embodiment of the invention. The offline learning stage 111 is performed prior to the online stage 112. The offline stage 111 consists of a learning system 120 which determines the motion bases vectors, which are stored 130 for use in the online step 112. The learning system 120 takes as input training data 110 in the form of images.

In other words, the offline stage is a computationally intensive step. The motion vector fields are determined for many example cases. i.e., from different patients based on historical data. After the motion fields are determined, some embodiments perform a dimensionality reduction on the motion vector fields, which can be used as basis vectors in the online step.

In particular, some embodiments aim to track tumors in anatomical areas of the body which are subject to breathing motion. At least one goal is to recover the dense motion fields that describe the pixel motion between consecutive frames of video. The modality considered is X-ray video, but it may be applicable to other modalities. Further, when determining the motion between consecutive images, i.e. a source and a target image, such an approach can be computationally expensive, and cannot be performed in real-time. Thus, to overcome such challenges, some embodiment introduces to identify a sparse set of landmarks in each of the consecutive two frames, and determine the correspondence of those landmarks between consecutive frames. These landmarks only give us local information, and we need to interpolate this local information to obtain motion information for all pixels in the source image. This interpolation can be performed according to previously learned information about the motion of breathing. This learning is performed using many example cases, in an offline step, which is stored historical data in memory. The example motion data is analyzed, and reduced to a much smaller set of key descriptive basis motions. These descriptive basis motions are then used in the online step to perform the interpolation of sparse landmarks correspondences.

In FIG. 1D the online stage 112 is comprised of a landmark detection system 150 to identify particular landmarks in the images from the imaging system 140. The imaging system 140 stores a previous image and a current image acquired close in time. The input to the landmark detection system 150 is the previous and current image pair from system 140. The output of the landmark detection system 150 together with the stored motion bases vectors 130 are input to the motion field reconstruction system 160, and its output is used to determine the tumor location 170 in a next image. The tumor location 170 is used as input to a controller 180, for example a controller to direct a radiotherapy system.

In other words, in the online step embodiments determine a set of sparse landmark points in consecutive video frames, and determine the correspondence of those landmarks. From the sparse set of corresponding landmark points and basis vectors, a dense motion vector field between the two frames is determined. The motion vectors in the dense field describe the motion for every pixel, and therefore also for the tumor. Whether the tumor is visible or not does not matter. This is the main advantage over previous approaches, which break down when the tumor is poorly or not visible. In addition, our invention allows for both markerless and marker-based tracking of a tumor, without any changes to the algorithm. Previous approaches are specifically designed for either markerless, or marker-based but cannot handle both cases. Another advantage is that the online step can be performed in real-time, i.e. 15 to 30 Hz, which is important for a practical application.

Offline Stage

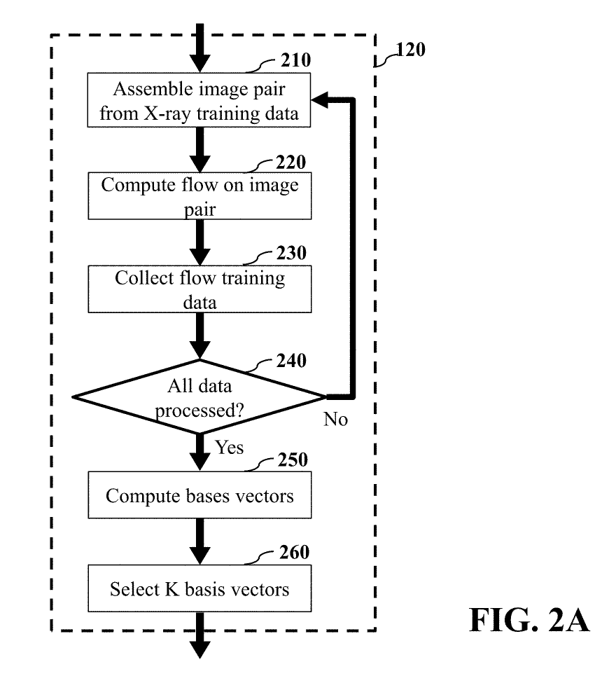

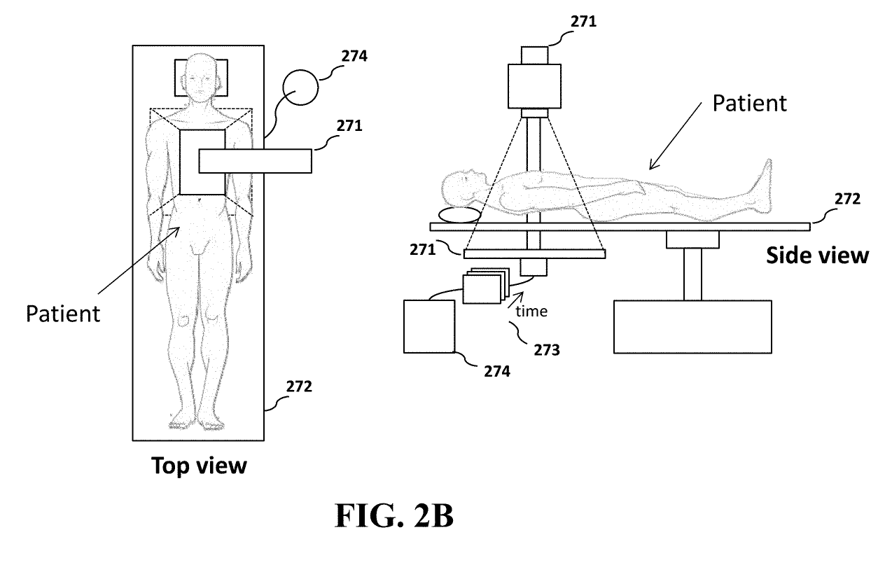

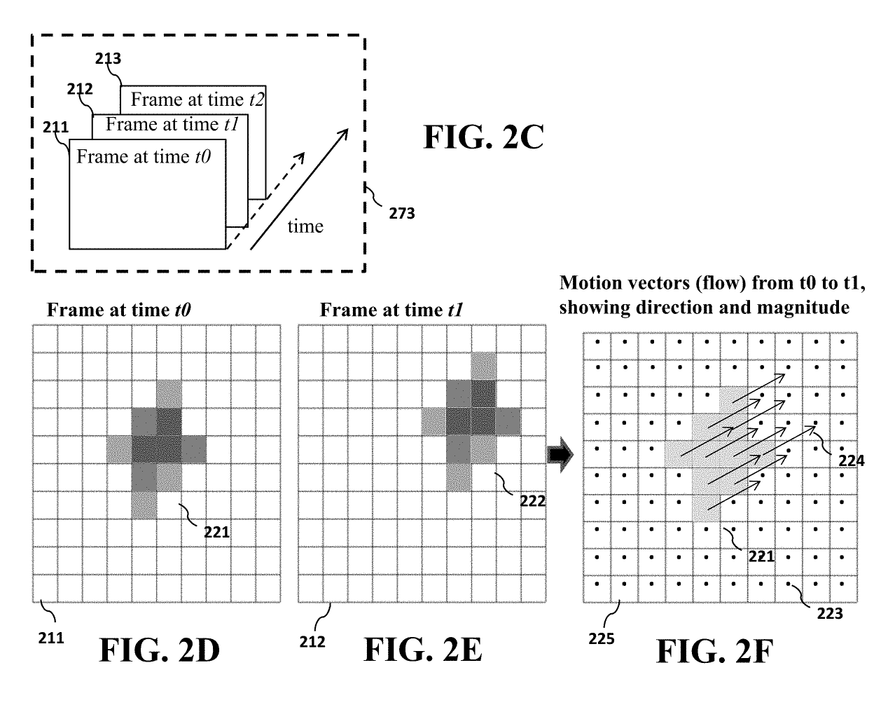

FIG. 2A shows a flow chart illustrating the steps involved to learn the motion bases vectors 120 according to one embodiment of the invention. The method takes as input images from the training data, and assembles a pair of images 210. The image pair is taken from consecutive frames in a video, or images acquired close in time. The method then computes a dense motion vector field (flow) 220 for the image pair. The computed motion vector field 220 is then added to the training data collection 230. The method decides whether all data is processed 240. If not, the methods will assemble a next training pair 210 and proceed. Once all training data is processed, the method computes a set of bases vectors 250, and determines a subset of K basis vectors 260 from the set of bases vectors 250, to be stored 130.

FIG. 2B is a top and side view schematic illustrating the concept of capturing of historical data of internal anatomical structures of patients, according to embodiments of the present disclosure. The patient is positioned on a table or treatment couch 272, and an X-ray imaging system 271 records a sequence 273 of X-ray images which are stored in memory 274.

FIG. 2C illustrates that a sequence 273 from FIG. 2B contains individual images 211, 212, 213 and so on. Individual image 211 is captured at time t0, whereas image 212 is captured at time t1, image 213 is captured at time t2, and so on. Time t0 of image 211 occurred earlier than time t1 of image 212, which itself occurred earlier than time t2 of image 213, etc.

FIGS. 2D and 2E illustrate a pair of temporally separated images, image 211 at time t0, and image 212 at time t1, which is later than time t0. Image 211 contains a region at a particular location 221. Image 212 contains the same region but at a different location 222.

FIG. 2F illustrates the motion vector field 225 for the images 211 and 212 of FIGS. 2D and 2E respectively. Given the locations 221 and 222 of a region, optical flow computes the motion vectors 223 and 224 in the motion vector field 225 for each pixel in image 211. Motion vectors 223 and 224 represent a direction and magnitude. Motion vectors 223 have magnitude equal to zero, indicating that for that particular pixel in image 211 no motion was detected between images 211 and 212. Motion vectors 224 indicate a direction and magnitude for the pixels of image 211 for which motion was detected between images 211 and 212.

In FIG. 2F the motion vector field 225 was determined from image 211 at time t0 to image 212 at time t1. Optical flow can produce a motion vector field from image 212 at time t1 to image 211 at time t0 as well. The ordering of time is not a requirement for optical flow computation. For optical flow computation it is only assumed that time t0 is not equal to time t1.

In order to better comprehend the offline training, the present disclosure can previously acquire K fluoroscopic videos, each fluoroscopic video has a total of nₖ+1 frames, with k∈[0,K−1]. For each video the present disclosure can compute a total of nₖ flow fields between the consecutive frames of the video. The total number of flow frames from all videos is N=n₀+ . . . +n_{K-1}. The flow fields can be computed using the Anisotropic Huber-L1 Optical Flow. The parameters can be empirically adjusted to obtain flow fields which both reduced the error between the warped source and target images, but still gave reasonably smooth flows.

Since N is typically very large, it is computationally prohibitive to use all N flow fields. Principal components analysis (PCA) can be used to reduce the dimensionality of the flow fields. PCA can be computed using eigen analysis, such that, projection of the data onto the eigenvector, corresponding to the largest eigenvalue gives the largest variance, projection onto eigenvector corresponding to second largest eigenvalue, gives the second largest variance, and so on, etc. The eigenvectors represent a set of bases vectors, and the original data can be represented as a weighted sum of the bases vectors:

$$u = {\sum }_{i = 1}^{M} {w}_{i} {b}_{i} . \qquad ( 1 )$$

In the case where M=N, the data is reconstructed exactly. Typically M<<N such that the reconstruction is approximate, where the cardinality of M determines the amount of reconstruction error.

PCA can be computed using the eigen analysis of the covariance matrix XX^{T}, obtained from the observation data×(the mean over all observations is first subtracted to obtain zero mean X). In the case of images, each image is stored in a memory as a 1-dimensional vector by concatenating each row of the image, and subsequently transpose it into a column vector. If an image has a total of P pixels, the covariance matrix would have dimension P×P. Performing eigen analysis on such a large matrix would be prohibitively expensive (if not impossible). Instead, an eigen analysis can be performed on x^{T}X, and compute the final eigenvectors as v=X*V. Then, it is possible to obtain a matrix A with the eigenvectors associated with the M largest eigenvalues, and each eigenvector represents a basis b in Equation 1 (Eq. 1). The images can be projected onto the principal component axes to obtain the PCA representation of each image Xₚₑₐ=A^{T}X.

The present disclosure uses a Linear PCA, of which, provides a efficient computational time. However, other methods can be used, such as using Grassman Averages which may be more robust against errors in the computed flow fields. In regard with the present disclosure, errors in the flow fields can result in errors in the flow bases. However, through experimentation, results were better using the linear PCA.

Prior to computing the flow fields over fluoroscopic video images, the present disclosure can first perform a robust subspace estimation to separate moving foreground components from the static background. In the case of X-ray images of the lungs, for example, the foreground component can contain tissues affected by respiratory motion. Whereas the background can mostly contain the spinal cord, ribs and bones. Robust subspace estimation decomposes the images of a video sequence into a low-rank component (the foreground), and a dense component (the background). The foreground component is a time-varying estimation adapting to the motion in the video. Once we have separated the frames of a video sequence into its foreground and background components, the flow can be computed on the foreground component, e.g., the moving lung tissue.

Online Stage

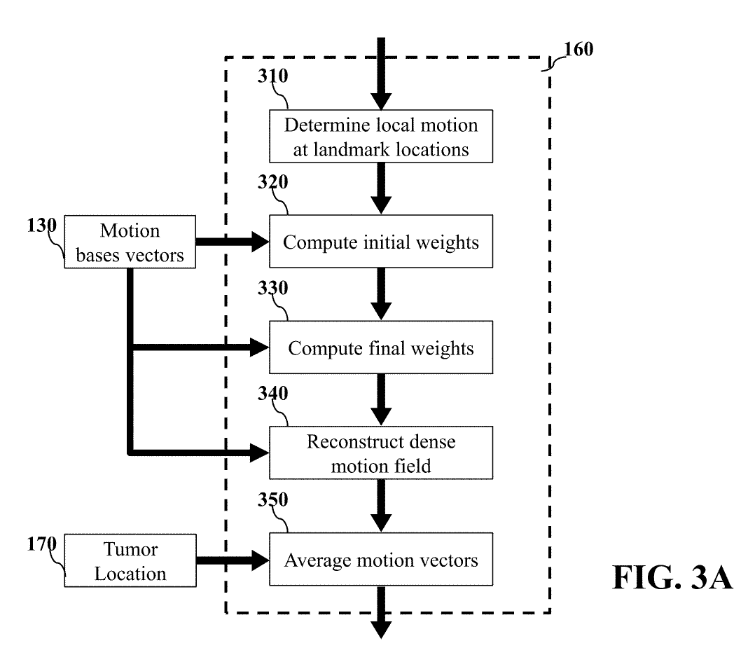

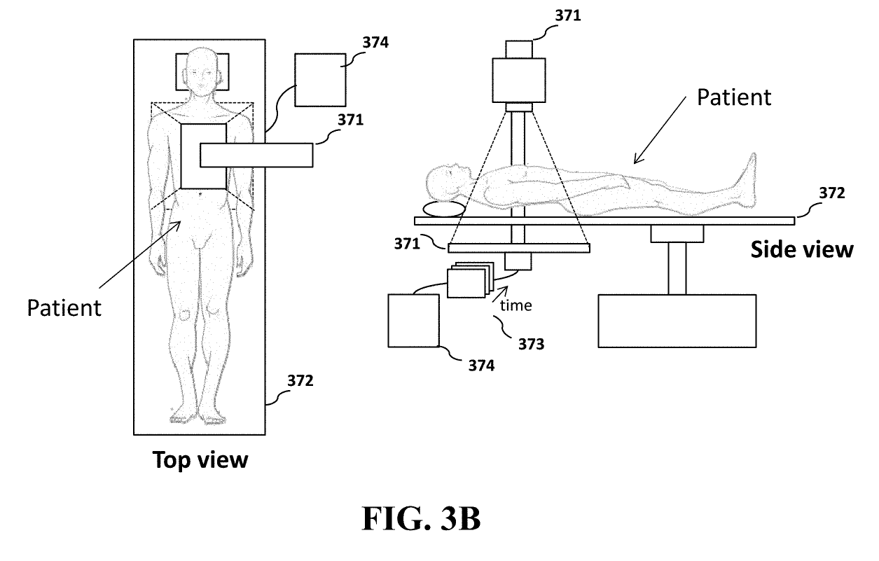

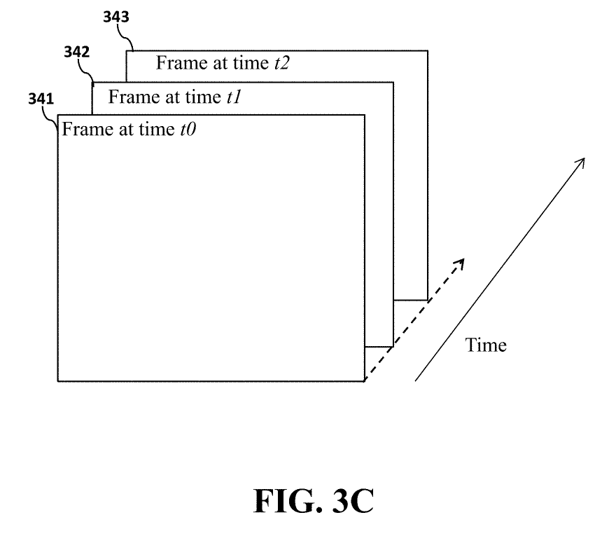

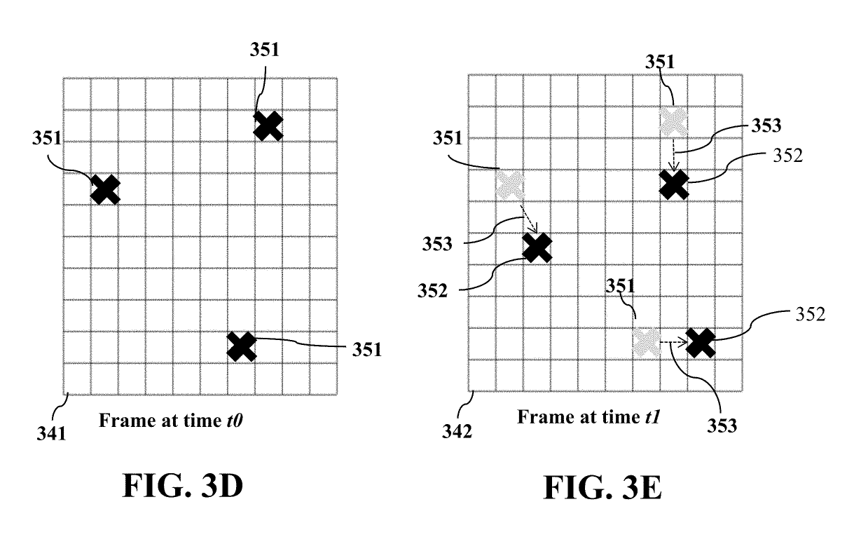

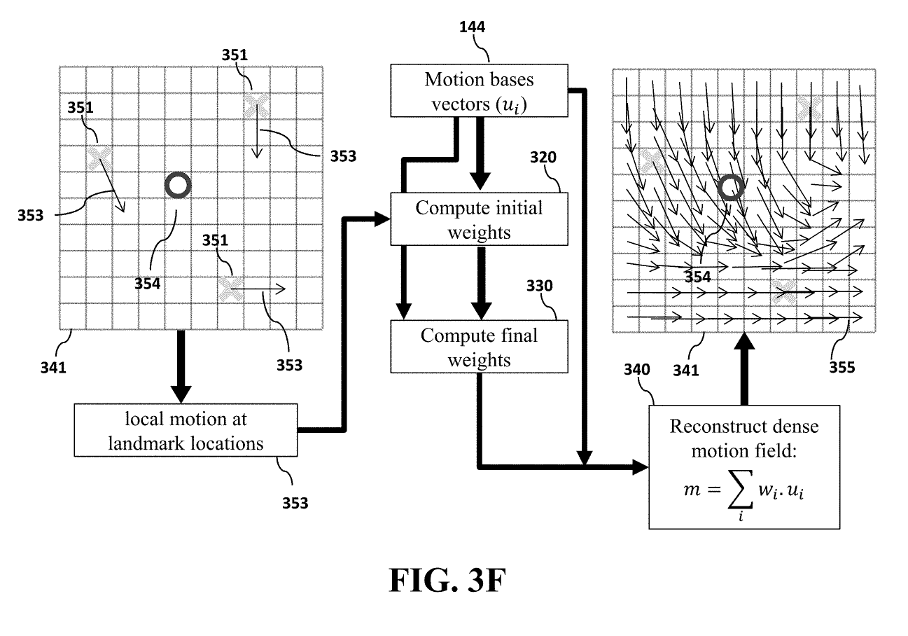

FIG. 3A is a block diagram illustrating some steps involved in the motion field reconstruction system to determine motion vectors for updating the tumor location, according to embodiments of the present disclosure.

FIG. 3A shows a flow chart illustrating the steps involved in the motion field reconstruction system 160 to determine motion vectors 350 for updating the tumor location 170 according to one embodiment of the invention. The method takes as input matched landmarks between a pair of images, which is the output of the landmark detection system 150. The method then computes the local motion at landmark locations 310 by subtracting the corresponding image locations. From the local motion at landmark locations 310 and motion bases vectors 130, a set of initial weights 320 is computed. The initial weights 320 and motion bases vectors 130 are then used to compute the final weights 330. A dense motion vector field 340 is then computed from the final weights 330 and motion bases vectors 130. Using a previous tumor location 170, the average motion vectors 350 are determined, which serve as input for updating the current tumor location 170.

In one embodiment of the present disclosure the reconstructed dense motion vector field 340 is computed as the weighted sum of motion bases vectors 130 where the weights are according to the final weights 330.

FIG. 3B is a top and side view schematic illustrating the concept of capturing online image data of internal anatomical structures of patients, according to embodiments of the present disclosure. The patient is positioned on a table or treatment couch 372, and an X-ray imaging system 371 provides a sequence 373 of X-ray images which are presented to a processor 374, which forms pairs of images from image in the sequence 373 for the duration of treatment.

FIG. 3C is a schematic illustrating the sequence 373 of FIG. 3B consisting of images 341 at time t0, 342 at time t1, and 343 at time t2 over time, according to embodiments of the present disclosure.

FIGS. 3D and 3E illustrate graphs of an initial pair of temporally separated images 341 at time t0, and image 342 at time t1. In image 341 at time t0 a number of landmarks 351 is determined by the landmark detection system 150. In image 342 at time t1 a number of landmarks 352 is determined by the landmark detection system 150. The landmark detection system 150 determines the landmark correspondence between landmarks 351 and 352. The landmark detection system detection system 150 determines the local motion information 353 for landmarks 351 using correspondence between landmarks 351 and 352.

Given a set of landmarks 351 with their associated local motion information 353, a motion for a region 354 should be determined from an interpolation of the local motion information. The interpolation is performed according to a dense motion field reconstruction. The local motion 353 at landmarks 351 and motion bases vectors 144 are used to compute initial weights 320 for combining the motion bases vectors 144. The initial weights 320, the motion vectors 144 and local motion information 353 at the landmarks 351 are used to determine the final weights 330. A reconstruction 340 then determines produces a dense motion vector field 341 with motion vectors 355 using the final weights 330 and motion vector bases 144.

According to one embodiment of the present disclosure the dense motion vector field 341 is reconstructed 340 as a weighted sum of the motion bases vectors 144 and the final weights 330 to produce motion vectors 355.

The dense motion vector field 341 now contains motion vectors 355 for a region 354.

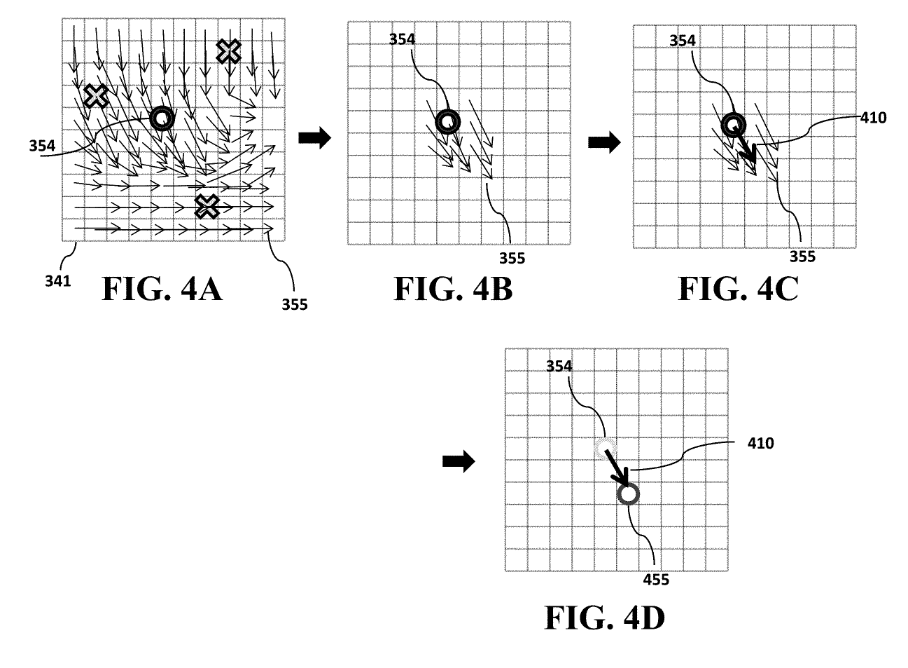

FIG. 4A shows the reconstructed motion vector field 341 with motion vectors 355 and a region 354. In FIG. 4B a neighborhood for region 354 is determined with information from motion vectors 355. The motion vectors 355 in neighborhood for region 354 are averaged to produce one or more motion vectors 410 in FIG. 4C. In FIG. 4D the motion vector 410 determines the location 455 for region 354 in a next image.

Online Feature Detection and Interpolation

To reconstruct the flow fields from the flow bases the present disclosure determines the weights w in Equation 1. We can determine these weights as follows. Given a set of matched features between consecutive frames, denoted as x, x′, we can determine local motion displacements at each pixel (or feature location): ū=x−x′. With the local displacements, we can solve for the weights in Equation 1 using linear least squares:

$$arg {min}_{w} P {\vert Aw - \hat{u} P \vert}_{2}^{2} . \qquad ( 2 )$$

Here A is a matrix of dimension M×m, with M the number of basis vectors as above, and m the number of feature matches. A is obtained by sampling each basis vector at the pixel location of each feature in the first image of each image pair. Given w, we use Equation 1 to reconstruct the dense flow field. Equation 2 is sensitive to outliers, i.e., incorrect matches, and we instead solve its robust version:

$$arg {min}_{w} ρ ( P {\vert Aw - \hat{u} P \vert}_{2}^{2} ) + λ P {\vert Γ wP \vert}_{2} . \qquad ( 3 )$$

Equation 3 can be solved efficiently using Iteratively Re-weighted Least Squares (IRLS). The function ρ( ) is a robust estimator, such as the Cauchy function. The regularization term, the second term in Equation 3, defines a prior on the weights. The prior is set as the inverse of the covariance of the training data projected onto the flow bases. This will cause bases with small influence to have small weights. Solving Equation 3 typically takes between 5 to 10 iterations of IRLS. We use the least squares solution of Equation 2 as the initial guess in IRLS.

Computing features for X-ray video image data is challenging since the images are grayscale, and they lack texture in some regions. Furthermore, we are dealing with transparencies in the imagery which potentially confuses the feature matching. We have chosen to compute features in the fluoroscopic images using SURF [?]. An example of detected features is shown in FIGS. 1(a) and (b). Although in some areas there are few to no features, these are areas where we do not need to reconstruct the flow field anyways, since they correspond to areas outside of the lung tissue.

During experimentation, the online step of the method may be executed in real-time, provided that feature detection is performed on GPU hardware. The X-ray images can be cropped to contain the region which spans the range of motion of the tumor. The feature detection using SURF and subsequent feature matching can be the most computationally expensive operations. Smaller input images can result in less candidate features that can be considered. IRLS may only takes 5 to 10 iterations, and with fewer features the update steps of IRLS can be computed quicker.

Further, the implanted fiducials can be directly taken into account as detected features. The corresponding weighting in matrix w in the IRLS update step (Eq. 3): \tilde{w}=(A^{T}WAû)⁻¹ A^{T}Wû can be adapted. By increasing the weighting of the reconstruction error for matched fiducial locations, through experimentation the present disclosure can ensure correct reconstruction near those fiducials

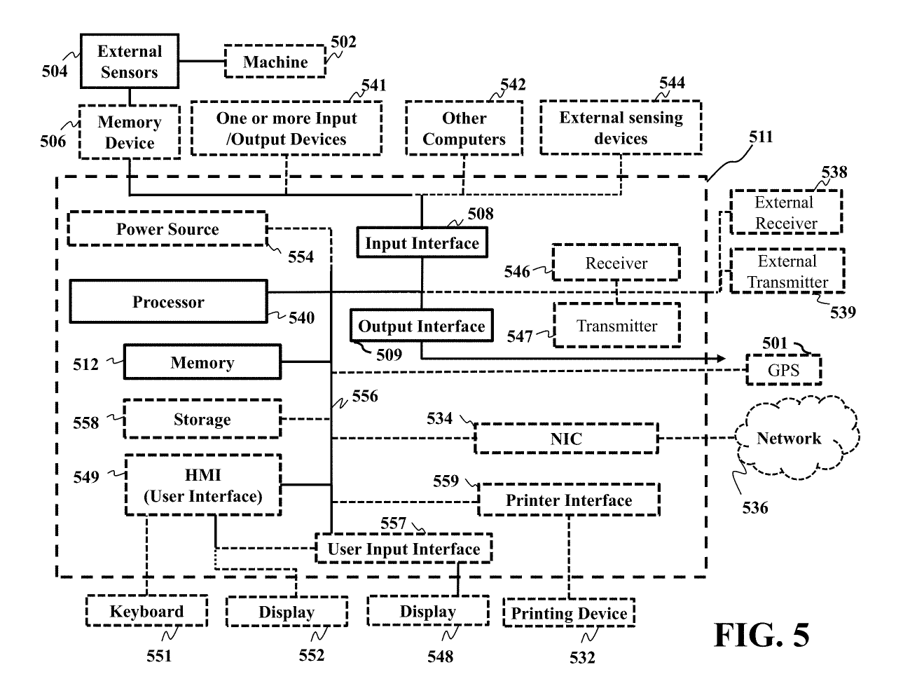

FIG. 5 is a block diagram illustrating the methods of FIG. 1A and FIG. 1B, that can be implemented using an alternate computer or processor, according to embodiments of the present disclosure.

FIG. 5 is a block diagram of illustrating the method of FIG. 1A, that can be implemented using an alternate computer or processor, according to embodiments of the present disclosure. The computer 511 includes a processor 540, computer readable memory 512, storage 558 and user interface 549 with display 552 and keyboard 551, which are connected through bus 556. For example, the user interface 564 in communication with the processor 540 and the computer readable memory 512, acquires and stores the signal data examples in the computer readable memory 512 upon receiving an input from a surface, keyboard surface 564, of the user interface 564 by a user.

The computer 511 can include a power source 554, depending upon the application the power source 554 may be optionally located outside of the computer 511. Linked through bus 556 can be a user input interface 557 adapted to connect to a display device 548, wherein the display device 548 can include a computer monitor, camera, television, projector, or mobile device, among others. A printer interface 559 can also be connected through bus 556 and adapted to connect to a printing device 532, wherein the printing device 532 can include a liquid inkjet printer, solid ink printer, large-scale commercial printer, thermal printer, UV printer, or dye-sublimation printer, among others. A network interface controller (NIC) 534 is adapted to connect through the bus 556 to a network 536, wherein time series data or other data, among other things, can be rendered on a third party display device, third party imaging device, and/or third party printing device outside of the computer 511.

Still referring to FIG. 5, the signal data or other data, among other things, can be transmitted over a communication channel of the network 536, and/or stored within the storage system 558 for storage and/or further processing. Contemplated is that the signal data could be initially stored in an external memory and later acquired by the processor to be processed or store the signal data in the processor's memory to be processed at some later time. The processor memory includes stored executable programs executable by the processor or a computer for performing the elevator systems/methods, elevator operation data, maintenance data and historical elevator data of the same type as the elevator and other data relating to the operation health management of the elevator or similar types of elevators as the elevator.

Further, the signal data or other data may be received wirelessly or hard wired from a receiver 546 (or external receiver 538) or transmitted via a transmitter 547 (or external transmitter 539) wirelessly or hard wired, the receiver 546 and transmitter 547 are both connected through the bus 556. The computer 511 may be connected via an input interface 508 to external sensing devices 544 and external input/output devices 541. For example, the external sensing devices 544 may include sensors gathering data before-during-after of the collected signal data of the elevator/conveying machine. For instance, environmental conditions approximate the machine or not approximate the elevator/conveying machine, i.e. temperature at or near elevator/conveying machine, temperature in building of location of elevator/conveying machine, temperature of outdoors exterior to the building of the elevator/conveying machine, video of elevator/conveying machine itself, video of areas approximate elevator/conveying machine, video of areas not approximate the elevator/conveying machine, other data related to aspects of the elevator/conveying machine. The computer 511 may be connected to other external computers 542. An output interface 509 may be used to output the processed data from the processor 540. It is noted that a user interface 549 in communication with the processor 540 and the non-transitory computer readable storage medium 512, acquires and stores the region data in the non-transitory computer readable storage medium 512 upon receiving an input from a surface 552 of the user interface 549 by a user.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine. Typically, the functionality of the program modules may be combined or distributed as desired in various embodiments.

Also, the embodiments of the present disclosure may be embodied as a method, of which an example has been provided. The acts performed as part of the method may be ordered in any suitable way. Accordingly, embodiments may be constructed in which acts are performed in an order different than illustrated, which may include performing some acts concurrently, even though shown as sequential acts in illustrative embodiments. Further, use of ordinal terms such as “first,” “second,” in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Although the present disclosure has been described with reference to certain preferred embodiments, it is to be understood that various other adaptations and modifications can be made within the spirit and scope of the present disclosure. Therefore, it is the aspect of the append claims to cover all such variations and modifications as come within the true spirit and scope of the present disclosure.

What is claimed is:

1. A real-time treatment system for tracking of an abnormal growth of tissue in a body of a patient over a treatment period, comprising the steps of:
a memory to store in an offline stage, historical image data of internal anatomical structures of bodies of different patients caused by a respiration of the patients;
a processor in communication with the memory during the offline state, is configured, to obtain for each patient image data, a sequence of dense motion vector fields, wherein the sequence of dense motion vector fields of all the patients is transformed into a set of learned bases vectors of breathing motion and stored in the memory;
an imaging interface of an online stage accepts an initial pair of temporally separated images of internal anatomical structures of the body of the patient during respiration in real-time;
an imaging processor in communication with the imaging interface, to track changes of positions of landmark points over the initial pair of temporally separated images, to produce a first local motion representation of the body of the patient, caused by the respiration of the patient;
an interpolation processor in communication with the memory and the imaging processor, interpolates the first local motion representation by accessing the memory, and using the stored set of learned bases vectors of breathing motion, to produce a first dense motion vector field of the body of the patient;
a controller in communication with interpolation processor, to track a first location of the abnormal growth of tissue of the patient, according to the produced first dense motion vector field, to produce a first location of the abnormal growth of tissue of the patient;
iteratively, accepting in real-time pairs of temporally separated images of internal anatomical structures of the body of the patient during respiration for each iteration, to produce a dense motion vector field of the body of the patient, to track a location of the abnormal growth of tissue of the patient from the first location, or a previously determined location, according to each produced dense motion vector field, and ending the iteration, at an end of the treatment period.

2. The system of claim 1, wherein the abnormal growth of tissue is a tumor.

3. The system of claim 1, wherein the interpolation processor performs the interpolation by determining a weighted combination of the bases vectors to produce the dense motion field approximating the local motion information.

4. The system of claim 3, wherein the weighted combination of the bases vectors is determined to provide an optimal fit of the local motion vectors.

5. The system of claim 4, wherein the optimal fit is computed by solving a least squares problem or by solving a robust least squares problem.

6. The system of claim 1, wherein the set of bases vectors is an output of a principal components analysis (PCA) on the dense motion vector fields.

7. The system of claim 6, wherein a subset of K basis vectors is chosen according to the K largest eigenvalues computed during PCA.

8. The system of claim 7, wherein the dense motion vector fields are determined using the Anisotropic Huber-L1 Optical Flow.

9. The system of claim 1, wherein the historical image data is transformed into a foreground component image that captures motion of anatomical structures in the image data and a background component image which captures static components in the image data.

10. The system of claim 1, wherein the dense motion vector fields are determined for foreground component images of the historical image data.

11. The system of claim 1, wherein the imaging processor tracks the changes of positions of the landmark points in the sequence of images by determining the correspondence of between the landmark points in consecutive pair of images, such that the images are X-ray images.

12. The system of claim 11, wherein the imaging processor detects and determines the landmark points using speeded up robust features (SURF) detector.

13. The system of claim 1, wherein the initial pair of temporally separated images in the online stage are transformed into a foreground component image that captures motion of anatomical structures in the image data and a background component image which captures static components in the image data.

14. The system of claim 13, wherein the imaging processor tracks the changes of positions of the landmark points in the sequence of foreground component images by determining the correspondence between the landmark points in a consecutive pair of foreground component images.

15. The system of claim 1, wherein the memory stores an initial location of the tumor, such that the controller tracks the location of the tumor according to the dense motion vector field starting from the initial location.

16. The system of claim 1, wherein the tracked location of the tumor is used to direct a radiotherapy system to irradiate the tumor at the current location, such that the radiotherapy system comprises a particle beam radiotherapy system.

17. A real-time treatment method, comprising the steps of:
storing in a memory in an offline stage, historical image data of internal anatomical structures of bodies of different patients caused by a respiration of the patients;
obtaining for each patient image data via a processor in communication with the memory during the offline stage, a sequence of dense motion vector fields, wherein the sequence of dense motion vector fields of all the patients is transformed into a set of learned bases vectors of breathing motion and stored in the memory, wherein the set of bases vectors is an output of a principal components analysis (PCA) on the dense motion vector fields;
accepting an initial pair of temporally separated images of internal anatomical structures of the body of the patient during respiration in real-time via an imaging interface of an online stage;
tracking changes of positions of landmark points over the initial pair of temporally separated images, to produce a first local motion representation of a body of a patient, caused by the respiration of the patient, via an imaging processor in communication with the imaging interface;
interpolating the first local motion representation by accessing the memory, and using the stored set of learned bases vectors of breathing motion, to produce a first dense motion vector field of the body of the patient over a treatment period, via an interpolation processor in communication with the memory and the imaging processor;
tracking a first location of an abnormal growth of tissue of the patient, according to the produced first dense motion vector field, to produce a first location of the abnormal growth of tissue of the patient, via a controller in communication with interpolation processor;
iteratively, accepting in real-time pairs of temporally separated images of internal anatomical structures of the body of the patient during respiration for each iteration, to produce a dense motion vector field of the body of the patient, to track a location of the abnormal growth of tissue of the patient from the first location, or a previously determined location, according to each produced dense motion vector field, and ending the iteration, at an end of the treatment period.

18. The method of claim 17, wherein the stored set of learned bases vectors is an output of a principal components analysis (PCA) on the sequence of dense motion vector fields reducing errors between warped source images and target images from the historical patient image data.

19. The method of claim 17, wherein the imaging processor tracks the changes of positions of the landmark points in the sequence of images by determining correspondence between the landmark points in consecutive pair of images.

20. The method of claim 19, wherein the imaging processor performs the interpolation by determining a weighted combination of the stored set of learned bases vectors, to produce the dense motion field approximating the local motion information.

21. A real-time treatment delivery system for tracking and irradiation of a tumor in a body of a patient over a treatment period, comprising:
a memory to store in an offline stage, historical patient image data of a breathing motion of bodies of different patients, caused by a respiration of the patients;
a processor in communication with the memory during the offline state, is configured, to obtain for each stored historical patient image data, a sequence of dense motion vector fields, wherein the sequence of dense motion vector fields of all the patients is transformed into a set of learned bases vectors of breathing motion, and stored in the memory;
an imaging interface of an online stage accepts an initial pair of temporally separated images of internal anatomical structures of the body of the patient positioned for the irradiation, during respiration in real-time;
an imaging processor in communication with the imaging interface, to track changes of positions of landmark points over the initial pair of temporally separated images, to produce a first local motion information of the body of the patient, caused by the respiration of the patient;
an interpolation processor in communication with the memory and the imaging processor, interpolates the first local motion information by accessing the memory, and using the stored set of learned bases vectors of breathing motion, to produce a first dense motion vector field of the body of the patient;
an accelerator to produce a particle beam suitable for radiotherapy of the patient; and
a controller in communication with interpolation processor, to track a first location of the tumor of the patient, according to the produced first dense motion vector field, to produce a first location of the tumor of the patient;
iteratively, accepting in real-time pairs of temporally separated images of internal anatomical structures of the body of the patient during respiration for each iteration, to produce a dense motion vector field of the body of the patient, to track a location of the tumor of the patient from the first location, or a previously determined location, according to each produced dense motion vector field, and ending the iteration, at an end of the treatment period.

22. The system of claim 21, wherein the local motion information includes local motion vectors, and the weighted combination of the stored set of learned bases vectors is determined to provide an optimal fit of the local motion vectors.
